(19) United States
(12) Patent Application Publication
(10) Pub. No.: US 2019/0059381 A1
(43) Pub. Date: Feb. 28, 2019

(54) METHOD FOR CONTROLLING PESTS OF SOYBEAN, CORN, AND COTTON PLANTS

(71) Applicant: BASF SE, Ludwigshafen am Rhein (DE)

(72) Inventors: Karsten KOERBER, Eppelheim (DE); Pascal BINDSCHAEDLER, Roemerberg (DE); Wolfgang VON DEYN, Neustadt (DE); Franz-Josef BRAUN, Durham, NC (US); Juergen LANGEWALD, Mannheim (DE); Jochen DIETZ, Karlsruhe (DE)

(21) Appl. No.: 16/076,965

(22) PCT Filed: Feb. 13, 2017
(86) PCT No.: PCT/EP2017/053132

§ 371 Date: Aug. 9, 2018

(30) Foreign Application Priority Data
Feb. 19, 2016 (EP) 16156598.1

(51) Int. Cl. A01N 43/80; C07D 261/04

(52) U.S. Cl. CPC A01N 43/80; C07D 261/04

(57) ABSTRACT

A method for controlling pests of soybean plants comprises the step of contacting the soybean plant, parts of it, its propagation material, the pests, their food supply, habitat or breeding grounds with one or more compounds of formula I

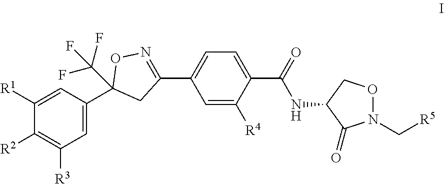

wherein the variables are defined as given in the description and claims.

The invention relates to a method for controlling pests of soybean, corn and cotton plants, comprising the step of contacting the plant, parts of it, its propagation material, the pests, their food supply, habitat or breeding grounds with one or more compounds of formula I

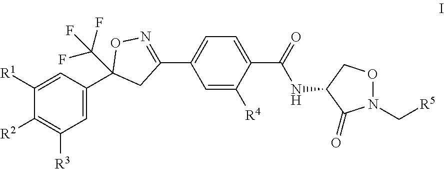

wherein;
R¹ is Cl or CF₃;
R² is F or H;
R³ is Cl or H;
R⁴ is CH₃ or Cl; and
R⁵ is H, CH₃ or CF₃,
   or a salt or N-oxide thereof.
   The above compounds can be used in pure form or as mixtures.

The compounds of formula I can be prepared by methods disclosed in the patent publication WO2011067272.

Faboideae, such as soybeans (Glycine max) and lima beans (Phaseolus lunatus), are important commercial crops.

Soybeans are considered to be a source of complete protein (Henkel, J., 2000, “Soy: Health Claims for Soy Protein, Question About Other Components”. FDA Consumer (Food and Drug Administration 34 (3): 18-20). For this reason, soy is a good source of protein. According to the US Food and Drug Administration, soy protein products can be good substitutes for animal products because soy offers a ‘complete’ protein profile. Soy protein products can replace animal-based foods which also have complete proteins but tend to contain more fat, especially saturated fat without requiring major adjustments elsewhere in the diet.

Soybean protein isolate is highly valuable as it has a biological value of 74 (Protein Quality Evaluation: Report of the Joint FAO/WHO Expert Consultation. Bethesda, Md. (USA): Food and Agriculture Organization of the United Nations (Food and Nutrition Paper No. 51, December 1989).

In agriculture soybeans can produce at least twice as much protein per acre than some other major vegetable or grain crop, e.g. 5 to 10 times more protein per acre than land set aside for grazing animals to make milk, and up to 15 times more protein per acre than land set aside for meat production (“Soy Benefits”, National Soybean Research Laboratory, February 2012).

Thus, soybeans can be regarded as a globally important crop providing oil and protein.

Nevertheless, soybean plants are vulnerable to a wide range of bacterial diseases, fungal diseases, viral diseases and parasites. Soybeans are considered to be e.g. the second-most valuable agricultural export in the United States behind corn.

Consequently, in view of the importance of soybean in agriculture, proper pest management is required in order not to jeopardize yield and quality of the soybean crops.

Stink bugs (order of Hemiptera, family of Pentatomidae) are animal pests and true bugs. They are probably one of the most common pest problems in soybean (Stewart et al., Soybean Insects—Stink bugs, University of Tennessee Institute of Agriculture, W200 09-0098).

Stink bugs feed on over 52 plants, including native and ornamental trees, shrubs, vines, weeds, and many cultivated crops such as corn and cotton, as well as numerous uncultivated plants, and their preferred hosts are nearly all wild plants. They build up on these hosts and move to soybeans late in the season as their preferred foods mature.

Stink bugs may feed on many parts of the plant; however, they typically target developing seed including the pods, meaning that injury to soybean seed is the primary problem associated with stink bug infestations.

Brown or blackish spots may occur where their mouthparts penetrate the plant tissue, but little external signs of feeding injury may be present. Feeding may cause deformation, shriveling or abortion of small seed. Larger seed may only be partly discolored by feeding injury, but this can affect seed quality. High levels of seed abortion may cause the “green bean effect” where foliage is retained and plant maturity is delayed (Stewart et al., Soybean Insects—Stink bugs, University of Tennessee Institute of Agriculture, W200 09-0098).

Stink bugs inflict mechanical injury to the seed as well as transmitting the yeast-spot disease organism. The degree of damage caused by this pest depends to some extent on the developmental stage of the seed when it is pierced by the stink bug's needlelike mouthparts. The younger the seed when damaged, the greater the yield reduction. Although late season infestations may not affect yield, bean oil content and germination will be reduced.

In certain regions the green stink bug (Acrosternum hilare) is one of the most common species that feeds on soybean. The brown stink bug (Euschistus servus) is another common component of the stink bug complex.

Of the complex of sucking bugs that occur in cultivation, the brown stinkbug Euschistus heros is currently considered to be the most abundant species in northern Parana to Central Brazil (Correa-Ferreira & Panizzi, 1999), and is a significant problem in soybean (Schmidt et al., 2003). The bugs occur in soybeans from the vegetative stage and are harmful from the beginning of pod formation until grain maturity. They cause damage to the seed (Galileo & Heinrichs 1978, Panizzi & Slansky Jr., 15, 1985) and can also open the way to fungal diseases and cause physiological disorders, such as soybean leaf retention (Galileo & Heinrichs 1978, Todd & Herzog, 1980).

Other plant feeding species that may be present include the red-shouldered stink bug (Thyanta custator) and the dusky-brown stink bug (Euschistus tristigmus). Another species, the southern green stink bug (Nezara viridula), is often confined to the southernmost counties of the US. Predatory (beneficial) stink bugs such as the spined soldier bug (Podisus maculaventris) may also be found in soybean and are sometimes mistaken for brown or dusky-brown stink bugs.

Control of stinkbugs in soybean is often vital to prevent significant economic damage.

Insecticides commonly used to control stinkbugs include pyrethroids, neonicotinoids and organophosphates, though pyrethroid insecticides are usually the method of choice for controlling stink bugs in soybean. However, there are increasing problems with insecticide resistance, particularly in brown stink bug populations and particularly to pyrethroids. Euschistus heros can also be difficult to manage using organophosphates or endosulfan (Sosa-Gomez et al., 2009). There is therefore a need for effective ecological methods of controlling stinkbugs in soybean.

Particularly insecticides acting on the gamma-aminobutyric acid (GABA)-gated chloride channel (disclosed in e.g. WO 2005/085216 (EP 1 731 512), WO 2009/002809 and WO 2009/080250) seem to be effective for controlling stinkbugs, especially in soybean such as described in WO2012/104331.

It has now been found that compounds of formula I provide an efficient control against pests on Faboideae, in particular soybeans, more particularly genetically modified soybeans, especially against pests from the families of Pentatomidae, and Thripidae.

These compounds therefore represent an important solution for controlling pests of Faboideae, in particular soybeans, more particularly genetically modified soybeans, in particular pests from the family of Pentatomidae, stink bugs, and thereby safeguarding plants, crops and propagation material from the infestation by such pests, particularly where the pests are resistant to current methods.

Compounds of formula I and their pesticidal activities on Egyptian cotton leafworm, Tobacco budworm, Diamond back moth, Corn rootworm, Green peach aphid, Onion thrips, and Twospotted spider mite, are disclosed in WO2011/067272.

None of these documents disclose an acceptable efficacy of compounds of formula I against typical pests of Faboideae, preferably soybeans, more preferably genetically modified soybeans, in particular against stink bugs. As stated above, these pests are difficult to control with typical soybean pesticides.

Accordingly, in one aspect of the invention there is provided a method for controlling pests of Faboideae, in particular soybean plants, comprising the step of contacting the Faboideae, in particular soybean, plant, parts of it, its propagation material, the pests, their food supply, habitat or breeding grounds with one or more compounds of formula I.

In a further aspect of the invention there is provided the use of one or more compounds of formula I for controlling pests in Faboideae, in particular soybean crops.

In a further aspect of the invention there is provided a method for controlling pests from the family of Pentatomidae and/or Thripidae, comprising the step of contacting the pests, their food supply habitat and/or breeding ground with one or more compounds of formula I.

In a further aspect of the invention there is provided a method for controlling pests from the family of Panicoideae plants, comprising the step of contacting the pests, their food supply habitat and/or breeding ground with one or more compounds of formula I and mixtures comprising compounds of formula I.

In a further aspect of the invention there is provided a method for controlling pests from the family of cotton plants, comprising the step of contacting the pests, their food supply habitat and/or breeding ground with one or more compounds of formula I and mixtures comprising compounds of formula I.

In yet a further aspect of the invention there is provided the use of one or more compounds of formula I for controlling pests of soybean, corn and cotton plants.

In one embodiment of the invention, the method comprises step of contacting the contacting the plant, parts of it, its propagation material, the pests, their food supply, habitat or breeding grounds a pesticidally effective amount of a compound of formula I which corresponds to formula I.1 or I.2

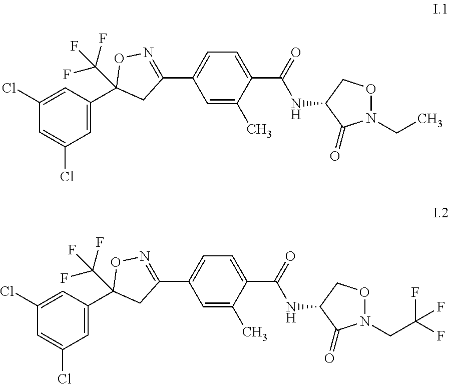 [SMILES: CCN1OC[C@@H](NC(=O)c2ccc(C3=NOC(c4cc(Cl)cc(Cl)c4)(C(F)(F)F)C3)cc2C)C1=O ; Cc1cc(C2=NOC(c3cc(Cl)cc(Cl)c3)(C(F)(F)F)C2)ccc1C(=O)N[C@@H]1CON(CC(F)(F)F)C1=O]

The compounds of formula (I) may exist in different geometric or optical isomers or tautomeric forms. This invention covers all such isomers and tautomers and mixtures thereof in all proportions as well as isotopic forms such as deuterated compounds.

The compounds of formula I may contain one or more asymmetric carbon atoms, and may exist as enantiomers (or as pairs of diastereoisomers) or as mixtures of such.

Depending on the substitution pattern, the compounds according to the invention may have one or more centers of chirality, in which case they are present as mixtures of enantiomers or diastereomers. The invention provides both the single pure enantiomers or pure diastereomers of the compounds according to the invention, and their mixtures and the use according to the invention of the pure enantiomers or pure diastereomers of the compounds according to the invention or their mixtures. Suitable compounds according to the invention also include all possible geometrical stereoisomers (cis/trans isomers) and mixtures thereof. Cis/trans isomers may be present with respect to an alkene, carbon-nitrogen double-bond or amide group. The term “stereoisomer(s)” encompasses both optical isomers, such as enantiomers or diastereomers, the latter existing due to more than one center of chirality in the molecule, as well as geometrical isomers (cis/trans isomers). The present invention relates to every possible stereoisomer of the compounds of formula I, i.e. to single enantiomers or diastereomers, as well as to mixtures thereof.

The compounds according to the invention may be amorphous or may exist in one or more different crystalline states (polymorphs) which may have different macroscopic properties such as stability or show different biological properties such as activities. The present invention relates to amorphous and crystalline compounds according to the invention, mixtures of different crystalline states of the respective compounds according to the invention, as well as amorphous or crystalline salts thereof.

Salts of the compounds according to the invention are preferably agriculturally and/or veterinary acceptable salts, preferably agriculturally acceptable salts. They can be formed in a customary manner, e.g. by reacting the compound with an acid of the anion in question if the compounds according to the invention have a basic functionality or by reacting acidic compounds according to the invention with a suitable base.

Agriculturally useful salts of the compounds according to the invention encompass especially the acid addition salts of those acids whose cations and anions, respectively, have no adverse effect on the pesticidal action of the compounds according to the invention.

Suitable cations are in particular the ions of the alkali metals, preferably Li, Na and K, of the alkaline earth metals, preferably Ca, Mg and Ba, and of the transition metals, preferably Mn, Cu, Zn and Fe, and also ammonium (NH₄) and substituted ammonium in which one to four of the H atoms are replaced by C₁-C₄-alkyl, C₁-C₄-hydroxyalkyl, C₁-C₄-alkoxy, C₁-C₄-alkoxy-C₁-C₄-alkyl, hydroxy-C₁-C₄-alkoxy-C₁-C₄-alkyl, phenyl or benzyl. Examples of substituted ammonium ions comprise methylammonium, isopropylammonium, dimethylammonium, diisopropylammonium, trimethylammonium, tetramethylammonium, tetraethylammonium, tetrabutylammonium, 2-hydroxyethylammonium, 2-(2-hydroxyethoxy)ethyl-ammonium, bis(2-hydroxyethyl)ammonium, benzyltrimethylammonium and benzyltriethylammonium, furthermore phosphonium ions, sulfonium ions, preferably tri(C₁-C₄-alkyl)sulfonium, and sulfoxonium ions, preferably tri(C₁-C₄-alkyl)sulfoxonium.

Anions of useful acid addition salts are primarily chloride, bromide, fluoride, hydrogensulfate, sulfate, dihydrogenphosphate, hydrogenphosphate, phosphate, nitrate, bicarbonate, carbonate, hexafluorosilicate, hexafluorophosphate, benzoate, and the anions of C₁-C₄-alkanoic acids, preferably formate, acetate, propionate and butyrate. They can be formed by reacting compounds according to the invention with an acid of the corresponding anion, preferably of hydrochloric acid, hydrobromic acid, sulfuric acid, phosphoric acid or nitric acid.

The term “N-oxide” includes any compound of the present invention which has at least one tertiary nitrogen atom that is oxidized to an N-oxide moiety.

The term “genetically modified plant” refers to a plant in which the genetic material has been altered in a way that does not occur naturally by mating and/or natural recombination. Examples of genetically modified plants of soybean are RR soybean and Intacta® soybean.

The methods and uses of the invention are for controlling and/or preventing infestation of Faboideae plants, Faboideae crops and Faboideae propagation material by pests. In one preferred embodiment, the Faboideae plants, crops or propagation material are soybean plants, crops or propagation material.

In another preferred embodiment, the Faboideae plants, crops or propagation material are lima bean plants, crops or propagation material.

In general, the pests are from the family of Pentatomidae and/or Thripidae. Preferably the methods and uses of the present invention are applied against pests from the family of Pentatomidae, stink bugs. More preferably against stink bugs that are resistant to other insecticides, e.g. pyrethroid insecticides. Stinkbugs that are “resistant” to a particular insecticide refers e.g. to strains of stinkbugs that are less sensitive to that insecticide compared to the expected sensitivity of the same species of stinkbug. The expected sensitivity can be measured using e.g. a strain that has not previously been exposed to the insecticide.

In an aspect of the invention, the method comprises applying to Faboideae plants, crops and/or propagation material, in particular soybean plants, soybean crops and/or propagation material of soybean plants or lima bean plants, lima bean crop and/or propagation material of lima bean plants, a compound of the invention, wherein the method is for controlling and/or preventing infestation by pests.

In another aspect of the invention, the method comprises applying to Panicoideae plants, crops and/or propagation material, in particular corn plants, corn crops and/or propagation material of corn plants, a compound of the formula I, wherein the method is for controlling and/or preventing infestation by pests.

In another aspect of the invention, the method comprises applying to Malvaceae plants, crops and/or propagation material, preferably cotton plants, in particular Bt cotton plants, Bt cotton crops and/or propagation material of Bt cotton plants, a compound of the formula I, wherein the method is for controlling and/or preventing infestation by pests.

Especially the method is for controlling and/or preventing infestation by pests from the family of Pentatomidae and/or Thripidae (such as Dichromothrips like Dichromothrips corbetti), in particular from the family of Pentatomidae, stink bugs; even more particular for controlling and/or preventing infestation by Acrosternum spp., Euschistus spp., Nezara spp. and/or Piezodrus spp., most particularly by Acrosternum hilare, Euschistus heros, Nezara viridula and/or Piezodrus guildini, and especially by Euschistus heros. Further Pentatomidae pests that can be controlled according to the invention are Eysarcoris, in particular Eysarcoris aeneus (forest shield bug). Further Heteroptera pests that can be controlled according to the invention include Miridae, such as Trigonotylus like Trigonotylus caelestialium (rice leaf bug).

A further aspect the invention provides the use of the compounds of the invention for the general control of pests from the family of Pentatomidae (stink bugs), Thripidae, preferably for the control of pests from the family of Pentatomidae, in particular for the control of Acrosternum spp., Euschistus spp., Nezara spp. and/or Piezodrus spp., more preferably for the control of Acrosternum hilare, Euschistus heros, Nezara viridula and/or Piezodrus guildini, and most preferably for the control of Euschistus heros.

In another aspect, the invention provides the use of the compounds of the invention for controlling pests that are resistant to one or more other insecticides, preferably pyrethroids, neonicotinoids and organophosphates, and more preferably pyrethroid insecticides.

Preferably the compounds of the invention are used for controlling pests from the family of Pentatomidae, stinkbugs, that are resistant to one or more other insecticides, preferably to pyrethroids, neonicotinoids and organophosphates, and more preferably to pyrethroid insecticides, in particular for the control of Acrosternum spp., Euschistus spp., Nezara spp. and/or Piezodrus spp., preferably for the control of Acrosternum hilare, Euschistus heros, Nezara viridula and/or Piezodrus guildini, and most preferably for the control of Euschistus heros that are resistant to one or more other insecticides, preferably pyrethroids, neonicotinoids and organophosphates, and more preferably pyrethroid insecticides.

The compounds applied in the methods of the invention may be used on Faboideae, Panicoideae and Malvaceae, including Bt cotton.

The compounds applied in the methods of the invention may be used on Faboideae, in particular soybean or lima beans, to control, any of: Elasmopalpus lignosellus, Euschistus variolarius, Euschistus impictiventris, Diloboderus abderus, Diabrotica speciosa, Sternechus subsignatus, Formicidae, Agrotis ipsilon, Julus ssp., Anticarsia gemmatalis, Megacopta spp., Megascelis ssp., Procornitermes ssp., Gryllotalpidae, Nezara viridula, Piezodorus spp., Acrosternum spp., Neomegalotomus spp., Cerotoma trifurcata, Popillia japonica, Edessa spp., Liogenys fuscus, Euschistus heros, stem borer, Dectes spp., stalk borer, Scaptocoris castanea, phyllophaga spp., Pseudoplusia includens, Spodoptera spp., Bemisia tabaci, Agriotes spp., Thripidae, preferably Diloboderus abderus, Diabrotica speciosa, Nezara viridula, Piezodorus spp., Acrosternum spp., Cerotoma trifurcata, Popillia japonica, Euschistus heros, phyllophaga spp., Agriotes spp., Spodoptera littoralis, Thrips spp., Spodoptera eridania, Helicoverpa sp., stinkbugs, and Dectes texanus.

The compounds of the invention are preferably used on Faboideae, in particular soybean or lima beans, to control stinkbugs, e.g. Nezara spp. (e.g. Nezara viridula, Nezara antennata, Nezara hilaris), Piezodorus spp. (e.g. Piezodorus guildinii), Acrosternum spp. (e.g. Acrosternum hilare), Euschistus spp. (e.g. Euschistus heros, Euschistus servus), Halyomorpha halys, Megacopta cribaria, Plautia crossota, Riptortus clavatus, Rhopalus msculatus, Antestiopsis orbitalus, Dectes texanus, Dichelops spp. (e.g. Dichelops furcatus, Dichelops melacanthus), Eurygaster spp. (e.g. Eurygaster intergriceps, Eurygaster maurd), Oebalus spp. (e.g. Oebalus mexicana, Oebalus poecilus, Oebalus pugnase, Scotinophara spp. (e.g. Scotinophara lurida, Scotinophara coarctatd). Preferred targets include Acrosternum hilare, Antestiopsis orbitalus, Dichelops furcatus, Dichelops melacanthus, Euschistus heros, Euschistus servus, Megacopta cribaria, Nezara viridula, Nezara hilare, Piezodorus guildinii, Halyomorpha halys. In one embodiment the stinkbug target is Nezara viridula, Piezodorus spp., Acrosternum spp., Euschistus heros. Euschistus and in particular Euschistus heros are the preferred targets.

The compounds applied in the methods of the invention may be used on Panicoideae in particular corn or sorghum or sugercane, to control, for example: Euschistus servus, Euschistus variolarius, Halyomorpha halys, Lygus hesperus, Lygus lineolaris, Lygus pratensis Spodoptera Frugiperta, Spodoptera exigua.

The compounds applied in the methods of the invention may be used on Malvaceae in particular cotton, including Bt cotton, or okra, to control, any of: Spodoptera littoralis, Thrips spp., Spodoptera eridania, Helicoverpa sp., and stinkbugs.

In a preferred embodiment of the invention the pests are from the family of Pentatomidae, Thripidae, or Miridae.

In another preferred embodiment of the invention the pests are selected from Acrosternum spp., Euschistus spp., Nezara spp., Piezodrus spp., Euschistus heros, Euschistus impictiventris, Euschistus variolarius, Euschistus servus, Nezara viridula, Piezodorus guildinii, Halyomorpha halys, Euschistus servus, Euschistus variolarius, Halyomorpha halys, Lygus hesperus, Lygus lineolaris, and Lygus pratensis.

In another preferred embodiment of the invention the pests are Acrosternum hilare, Euschistus heros, Nezara viridula, and/or Piezodorus guildini.

Further Pentatomidae pests that can be controlled according to the invention are Eysarcoris, in particular Eysarcoris aeneus.

The compounds applied in the methods of the invention may be used on Panicoideae, in particular corn, to control, any of: Euschistus servus, Euschistus variolarius, Halyomorpha halys, Lygus hesperus, Lygus lineolaris, Lygus pratensis, Spodoptera Frugiperta, and Spodoptera exigua.

In some embodiments, the invention relates to methods and uses, wherein the compound of the formula (I) is applied in an application type which corresponds in each case to one row of Table A.

In some embodiments, the invention relates to methods and uses, wherein the compound of the formula (I) as component I and at least one mixing partner as defined below, are applied in an application type which corresponds in each case to one row of Table A.

TABLE A

| Appl. | | |
|---|---|---|
| type | Crop | Pest |
| A-1 | Soybean | Spodoptera littoralis |
| A-2 | Soybean | Anticarsia gemmatalis |
| A-3 | Soybean | Spodoptera exigua |
| A-4 | Soybean | Helicoverpa sp. |
| A-5 | Soybean | Spodoptera eridania |
| A-6 | Soybean | Nezara spp. |
| A-7 | Soybean | Piezodorus spp. |
| A-8 | Soybean | Acrosternum spp. |
| A-9 | Soybean | Euschistus spp. |
| A-10 | Soybean | Halyomorpha halys |
| A-11 | Soybean | Megacopta cribaria |
| A-12 | Soybean | Plautia crossota |
| A-13 | Soybean | Riptortus clavatus |
| A-14 | Soybean | Rhopalus msculatus |
| A-15 | Soybean | Antestiopsis orbitalus |
| A-16 | Soybean | Dectes texanus |
| A-17 | Soybean | Dichelops spp. |
| A-18 | Soybean | Eurygaster spp. |
| A-19 | Soybean | Oebalus spp. |
| A-20 | Soybean | Scotinophara spp. |
| A-21 | Corn | Spodoptera |
| | | Frugiperta |
| A-22 | Corn | Euschistus servus |
| A-23 | Corn | Euschistus variolarius |
| A-24 | Corn | Halyomorpha halys |
| A-25 | Corn | Lygus hesperus |
| A-26 | Corn | Spodoptera exigua |
| A-27 | Cotton | Spodoptera littoralis |
| A-28 | Cotton | Thrips spp. |
| A-29 | Cotton | Spodoptera eridania |
| A-30 | Cotton | Helicoverpa sp. |

Application of the compounds of the invention is preferably to a crop of Faboideae, such as soybean or lima bean plants; panicoideae such as corn; and cotton such as Bt cotton in combination with Lygus species, the locus thereof or propagation material thereof. Preferably application is to a crop of Faboideae, such as soybean or lima bean plants or the locus thereof, more preferably to a crop of soybean plants. Application may be before infestation or when the pest is present. Application of the compounds of the invention can be performed according to any of the usual modes of application, e.g. foliar, drench, soil, in furrow etc. Control of stinkbugs can be achieved by foliar application, which is a preferred mode of application according to the invention.

In another preferred embodiment, the compounds of the invention are applied to Faboideae crops by soil-drench application. In one preferred embodiment, the Faboideae crops are soybean crops. In another preferred embodiment the Faboideae crops are lima bean crops.

In a further preferred embodiment the compounds of the invention are applied as seed-treatment to seeds of Faboideae, Faboideae, Panicoideae and Bt cotton crops. In one preferred embodiment, the Faboideae crops are soybean crops, particularly Bt soybean crops. In another preferred embodiment the Faboideae crops are lima bean crops.

The pest, e.g. the stink bugs, the plant, soil or water in which the plant is growing can be contacted with the compounds of the invention or composition(s) containing them by any further application method known in the art. As such, “contacting” includes both direct contact (applying the compounds/compositions directly on the animal pest or plant—typically to the foliage, stem or roots of the plant) and indirect contact (applying the compounds/compositions to the locus of the animal pest or plant).

The compounds of the invention or the pesticidal compositions comprising them may be used to protect growing plants and crops from attack or infestation by animal pests, especially from stink bugs, in particular from Euschistus, more particularly from E. heros, by contacting the plant/crop with a pesticidally effective amount of compounds of the invention. The term “crop” refers both to growing and harvested crops.

The compounds of the invention may be applied in combination with an attractant. An attractant is a chemical that causes the insect to migrate towards the location of application. For control of stinkbugs it can be advantageous to apply the compounds of the invention with an attractant, particularly when the application is foliar. Stinkbugs are often located near to the ground, and application of an attractant may encourage migration up the plant towards the active ingredient.

Suitable attractants include glucose, sacchrose, salt, glutamate, citric acid, soybean oil, peanut oil and soybean milk. Glutamate and citric acid are of particular interest, with citric acid being preferred.

An attractant may be premixed with the compound of the invention prior to application, e.g. as a readymix or tankmix, or by simultaneous application or sequential application to the plant. Suitable rates of attractants are for example 0.02 kg/ha-3 kg/ha.

The compounds of the invention are preferably used for pest control on Faboideae, in particular soybean or lima beans, at 1-500 g/ha, preferably 10-100 g/ha.

The compounds of the invention are suitable for use on any Faboideae plant, such as soybean plants or lima bean plants, including those that have been genetically modified to be resistant to active ingredients such as herbicides or to produce biologically active compounds that control infestation by plant pests.

In a further preferred embodiment, transgenic plants and plant cultivars obtained by genetic engineering methods, if appropriate in combination with conventional methods (Genetically Modified Organisms), and parts thereof, are treated. Particularly preferably, plants of the plant cultivars which are in each case commercially available or in use are treated according to the invention. Plant cultivars are understood as meaning plants having novel properties (“traits”) which have been obtained by conventional breeding, by mutagenesis or by recombinant DNA techniques.

These can be cultivars, bio- or genotypes. Depending on the plant species or plant cultivars, their location and growth conditions (soils, climate, vegetation period, diet), the treatment according to the invention may also result in superadditive “synergistic”) effects.

Thus, for example, reduced application rates and/or a widening of the activity spectrum and/or an increase in the activity of the substances and compositions which can be used according to the invention, better plant growth, increased tolerance to high or low temperatures, increased tolerance to drought or to water or soil salt content, increased flowering performance, easier harvesting, accelerated maturation, higher harvest yields, higher quality and/or a higher nutritional value of the harvested products, better storage stability and/or processability of the harvested products are possible, which exceed the effects which were actually to be expected.

The preferred transgenic plants or plant cultivars (obtained by genetic engineering) which are to be treated according to the invention include all plants which, by virtue of the genetic modification, received genetic material which imparts particularly advantageous, useful traits to these plants.

Examples of such traits are better plant growth, increased tolerance to high or low temperatures, increased tolerance to drought or to water or soil salt content, increased flowering performance, easier harvesting, accelerated maturation, higher harvest yields, higher quality and/or a higher nutritional value of the harvested products, better storage stability and/or processability of the harvested products.

Further and particularly emphasized examples of such traits are a better defense of the plants against animal and microbial pests, such as against insects, mites, phytopathogenic fungi, bacteria and/or viruses, and also increased tolerance of the plants to certain herbicidally active compounds.

Traits that are emphasized in particular are the increased defense of the plants against insects, arachnids, nematodes and slugs and snails by virtue of toxins formed in the plants, in particular those formed in the plants by the genetic material from Bacillus thuringiensis (for example by the genes CryIA(a), CryIA(b), CryIA(c), CryIIA, CryIIIA, CryIIIB2, Cry9c, Cry2Ab, Cry3Bb and CryIF and also combinations thereof) (referred to herein as “Bt plants”). Traits that are also particularly emphasized are the increased defense of the plants against fungi, bacteria and viruses by systemic acquired resistance (SAR), systemin, phytoalexins, elicitors and resistance genes and correspondingly expressed proteins and toxins.

Traits that are furthermore particularly emphasized are the increased tolerance of the plants to certain herbicidally active compounds, for example imidazolinones, sulphonylureas, glyphosate or phosphinotricin (for example the “PAT” gene). The genes which impart the desired traits in question can also be present in combination with one another in the transgenic plants.

Examples of “Bt plants” are soybean varieties which are sold under the trade names YIELD GARD®, and INTACTA®.

Examples of herbicide-tolerant plants which may be mentioned are soya bean varieties which are sold under the trade names Roundup Ready® (tolerance to glyphosate), Liberty Link® (tolerance to phosphinotricin), IMI® (tolerance to imidazolinones) and STS® (tolerance to sulphonylureas). An example of a glyphosate tolerant soybean cultivar is BMX Potencia.

Herbicide-resistant plants (plants bred in a conventional manner for herbicide tolerance) which may be mentioned include the varieties sold under the name Clearfield® (for example maize).

In another preferred embodiment, the compounds of the invention are applied to Faboideae, in particular soybean plants, carrying two or more traits (e.g. Enlist®), glyphosate (e.g. Roundup Ready®, Roundup Ready 2 Yield®), sulfonylurea (e.g. STS®), glufosinate (e.g. Liberty Link®, Ignite®), Dicamba (Monsanto) HPPD tolerance (e.g. isoxaflutole herbicide) (Bayer CropScience, Syngenta). Double or triple stack in soybean plants of any of the traits described here are also of interest, including glyphosate and sulfonyl-urea tolerance (e.g. Optimum GAT®, plants stacked with STS® and Roundup Ready® or Roundup Ready 2 Yield®), dicamba and glyphosate tolerance (Monsanto). Soybean Cyst Nematode resistance soybean (SCN®—Syngenta) and soybean with Aphid resistant trait (AMT®—Syngneta) are also of interest.

These statements also apply to plant cultivars having these genetic traits or genetic traits still to be developed, which plant cultivars will be developed and/or marketed in the future.

The present invention also relates to a method for controlling pests and/or increasing the plant health of a cultivated plant as compared to the respective non-modified control plant, comprising the application of the compound of the formula (I), to a plant with at least one modification, parts of such plant, plant propagation material, or at its locus of growth, wherein the cultivated plant is

(i) Gossypium hirsutum L. (cotton) or Zea mays L. (maize) and comprises at least one gene selected from the group consisting of dvsnf7, cry1A, cry1Ab-Ac, cry1C, cry2Ab2, cry2Ae, mocry1F; or

(ii) Glycine max L. (soybean), Triticum aestivum (wheat) or Oryza sativa L. (rice) and comprises at least one gene selected from the group consisting of cry34Ab1, cry35 Ab1, cry3A, cry3Bbl, dvsnf7, mcry3A, cry1A, cry1A.105, cry1Ab, cry1Ab-Ac, cry1Ac, cry1C, cry1F, cry1Fa2, cry2Ab2, cry2Ae, cry9c, mocry1F, pinII, vip3A(a), vip3Aa20.

Prefarably, present invention also relates to a method for controlling pests and/or increasing the plant health of a cultivated plant as compared to the respective non-modified control plant, comprising the application of the compound of the formula (I), to a plant with at least one modification, parts of such plant, plant propagation material, or at its locus of growth, wherein the cultivated plant is modified by at least one gene according to one row of table D.

TABLE D

| No. | crop | gene |
|---|---|---|
| D-1 | cotton | dvsnf7 |
| D-2 | cotton | cry1A |
| D-3 | cotton | cry1Ab-Ac |
| D-4 | cotton | cry1C |
| D-5 | cotton | cry2Ab2 |
| D-6 | cotton | cry2Ae |
| D-7 | cotton | mocry1F |
| D-8 | maize | dvsnf7 |
| D-9 | maize | cry1A |
| D-10 | maize | cry1Ab-Ac |
| D-11 | maize | cry1C |
| D-12 | maize | cry2Ab2 |
| D-13 | maize | cry2Ae |
| D-14 | maize | mocry1F |
| D-15 | soybean | cry34Ab1 |
| D-16 | soybean | cry35 Ab1 |
| D-17 | soybean | cry3A |
| D-18 | soybean | cry3Bb1 |
| D-19 | soybean | dvsnf7 |
| D-20 | soybean | mcry3A |
| D-21 | soybean | cry1A |
| D-22 | soybean | cry1A.105 |
| D-23 | soybean | cry1Ab |
| D-24 | soybean | cry1Ab-Ac |
| D-25 | soybean | cry1Ac |
| D-26 | soybean | cry1C |
| D-27 | soybean | cry1F |
| D-28 | soybean | cry1Fa2 |
| D-29 | soybean | cry2Ab2 |
| D-30 | soybean | cry2Ae |
| D-31 | soybean | cry9c |
| D-32 | soybean | mocry1F |
| D-33 | soybean | pinII |
| D-34 | soybean | vip3A(a) |
| D-35 | soybean | vip3Aa20 |

The present invention also relates to a mixture of at least one compound of the present invention with at least one mixing partner as defined herein after. Preferred are binary mixtures of one compound of the present invention as component I with one mixing partner as defined herein after as component II. Preferred weight ratios for such binary mixtures are from 5000:1 to 1:5000, preferably from 1000:1 to 1:1000, more preferably from 100:1 to 1:100, particularly preferably from 10:1 to 1:10. In such binary mixtures, components I and II may be used in equal amounts, or an excess of component I, or an excess of component II may be used.

Mixing partners can be selected from pesticides, in particular insecticides, nematicides, and acaricides, fungicides, herbicides, plant growth regulators, fertilizers, and the like. Preferred mixing partners are insecticides, nematicides and fungicides.

The following list M of pesticides, grouped and numbered according the Mode of Action Classification of the Insecticide Resistance Action Committee (IRAC), together with which the compounds of the present invention can be used and with which potential synergistic effects might be produced, is intended to illustrate the possible combinations, but not to impose any limitation:

M.1. GABA-gated chloride channel antagonists such as: fiproles (phenylpyrazoles): ethiprole, fipronil, or pyriprole;

M.2. Sodium channel modulators from the class of pyrethroids: bifenthrin, lambda-cyhalothrin, alpha-cypermethrin or deltamethrin;

M.3. Nicotinic acetylcholine receptor agonists (nAChR) from the class of neonicotinoids: clothianidin, dinotefuran, imidacloprid, thiacloprid, or thiamethoxam;

M.4. Nicotinic acetylcholine receptor allosteric activators from the class of spinosyns: spinosad;

M.5. Chloride channel activators from the class of avermectins and milbemycins: abamectin, emamectin benzoate, or ivermectin;

M.6. Selective homopteran feeding blockers: pymetrozine, or flonicamid;

M.7. Uncouplers of oxidative phosphorylation via disruption of the proton gradient: chlorfenapyr;

M.8. Inhibitors of the chitin biosynthesis type 0, such as benzoylureas: teflubenzuron;

M.9. Inhibitors of the chitin biosynthesis type 1: buprofezin;

M.10. Ecdyson receptor agonists such as diacylhydrazines: tebufenozide;

M.11. Octopamin receptor agonists: amitraz;

M.12. Voltage-dependent sodium channel blockers: metaflumizone;

M.13. Inhibitors of the of acetyl CoA carboxylase, such as Tetronic and Tetramic acid derivatives: spirotetramat;

M.14. Ryanodine receptor-modulators from the class of diamides: flubendiamide, chlorantraniliprole (Rynaxypyr®), cyantraniliprole (Cyazypyr®), tetraniliprole, or cyclaniliprole;

M.15. insecticidal active compounds of unknown or uncertain mode of action: afidopyropen, broflanilide, flupyradifurone, or sulfoxaflor;

M.16. ecdysone.

The commercially available compounds of the group M listed above may be found in The Pesticide Manual, 16th Edition, C. MacBean, British Crop Protection Council (2013) among other publications. The online Pesticide Manual is updated regularly and is accessible through http://bcpcdata.com/pesticide-manual.html.

Another online data base for pesticides providing the ISO common names is http://www.alanwood.net/pesticides.

The M.3 neonicotinoid cycloxaprid is known from WO2010/069266 and WO2011/069456, the neonicotinoid M.3, sometimes also to be named as guadipyr, is known from WO2013/003977, and the neonicotinoid M.3 (approved as paichongding in China) is known from WO2007/101369. The metaflumizone analogue M.12 is described in CN10171577 and the analogue in CN102126994. The phthalamides M.14 are known from WO2007/101540. The anthranilamide M.14 is described in WO2005/077934. The broflanilide are described in WO2010/018714.

In one preferred embodiment the mixing partner is selected from ethiprole, fipronil or pyriprole from group M.1; lambda cyhalothrin, alpha-cypermethrin, bifenthrin or deltamethrin from group M.2; dinotefuran, clothianidin, thiacloprid, imidacloprid or thiamethoxam from group M.3; spinosad from group M.4; ivermectin, abamectin, avermectin or emamectin from group M.5; flonicamid, pymetrozine from group M.6; chlorfenapyr from group M.7; teflubenzuron from group M.8; buprofezin from group M.9; tebufenozide from group M.10; amitraz from group M.11; metaflumizone from group M.12; spirotetramat from group M.13; flubendiamide, chlorantraniliprole, cyantraniliprole, tetraniliprole or cyclaniliprole from group M.14; afidopyropen, broflanilide, sulfoxaflor or flupyradifuron from group M.15; ecdysone from group M.16.

In another preferred embodiment, the mixing partner is selected from pyrethroids for example lambda cyhalothrin, alpha-cypermethrin, bifenthrin and deltamethrin from group M.2; or neonicotinoids for example dinotefuran, clothianidin, thiacloprid, imidacloprid or thiamethoxam from group M.3.

In still another preferred embodiment, the mixing partner is particularly selected from pyrethroids for example lambda cyhalothrin, alpha-cypermethrin, bifenthrin and deltamethrin from group M.2.

Insecticidal compositions for use typically contain from 0.001 to 95 weight %, preferably from 0.1 to 45 weight %, and more preferably from 1 to 25 weight % of at least one repellent and/or insecticide.

Ready-to-use preparations contain the compounds acting against parasites, preferably ectoparasites, in concentrations of 10 ppm to 80 percent by weight, preferably from 0.1 to 65 percent by weight, more preferably from 1 to 50 percent by weight, most preferably from 5 to 40 percent by weight.

Preparations which are diluted before use contain the compounds acting against ectoparasites in concentrations of 0.5 to 90 percent by weight, preferably of 1 to 50 percent by weight.

The compounds of the invention may be applied in the methods of the present invention in mixtures with fertilizers (for example nitrogen-, potassium- or phosphorus-containing fertilizers). Suitable formulation types include granules of fertilizer. The mixtures preferably contain up to 25% by weight of the compound of the invention.

An additional mixing partner may provide a composition having a broader spectrum of activity or increased persistence at a locus; synergize the activity or complement the activity (for example by increasing the speed of effect or overcoming repellency) of the compound of the invention; or help to overcome or prevent the development of resistance to individual components.

The particular additional active ingredient will depend upon the intended utility of the composition.

The compounds of the invention may be mixed with soil, peat or other rooting media for the protection of plants against seed-borne, soil-borne or foliar fungal diseases.

Examples of suitable synergists for use in the compositions include piperonyl butoxide, sesamex, safroxan, and dodecyl imidazole.

Preferred methods and uses of the invention applying the compound of formula I to a plant selected from RR soybean, Intacta® soybean, and Bt Cotton, particularly to Intacta® soybean. Particularly preferred methods are directed to applying the compound of formula I to plants and pests are given in Table 1 and Table 2.

Table 1 Methods and uses, wherein the compound I.1 is applied to plant and/or pests which corresponds in each case to one row of Table T.

Table 2 Methods and uses, wherein the compound I.2 is applied to plant and/or pests which corresponds in each case to one row of Table T.

TABLE T

| No. | Plant | Pest |
|---|---|---|
| T-1 | RR Soybean | Euschistus heros |
| T-2 | RR Soybean | Euschistus impictiventris |
| T-3 | RR Soybean | Euschistus variolarius |
| T-4 | RR Soybean | Euschistus servus |
| T-5 | RR Soybean | Nezara viridula |
| T-6 | RR Soybean | Piezodorus guildinii |
| T-7 | RR Soybean | Halyomorpha halys |
| T-8 | Intacta ® soybean | Euschistus heros |
| T-9 | Intacta ® soybean | Euschistus impictiventris |
| T-10 | Intacta ® soybean | Euschistus variolarius |
| T-11 | Intacta ® soybean | Euschistus servus |
| T-12 | Intacta ® soybean | Nezara viridula |
| T-13 | Intacta ® soybean | Piezodorus guildinii |
| T-14 | Intacta ® soybean | Halyomorpha halys |
| T-15 | Corn | Euschistus servus |
| T-16 | Corn | Euschistus variolarius |
| T-17 | Corn | Halyomorpha ha |
| T-18 | Corn | Lygus hesperus |
| T-19 | Corn | Lygus lineolaris |
| T-20 | Corn | Lygus pratensis |
| T-21 | Bt Cotton | Spodoptera littoralis |
| T-22 | Bt Cotton | Thrips spp. |
| T-23 | Bt Cotton | Spodoptera eridania |
| T-24 | Bt Cotton | Helicoverpa sp. |

The invention also relates to agrochemical compositions comprising an auxiliary and at least one compound of the present invention or a mixture thereof.

An agrochemical composition comprises a pesticidally effective amount of a compound of the present invention or a mixture thereof. The term “pesticidally effective amount” is defined below.

The compounds of the present invention or the mixtures thereof can be converted into customary types of agro-chemical compositions, e. g. solutions, emulsions, suspensions, dusts, powders, pastes, granules, pressings, capsules, and mixtures thereof. Examples for composition types are suspensions (e.g. SC, OD, FS), emulsifiable concentrates (e.g. EC), emulsions (e.g. EW, EO, ES, ME), capsules (e.g. CS, ZC), pastes, pastilles, wettable powders or dusts (e.g. WP, SP, WS, DP, DS), pressings (e.g. BR, TB, DT), granules (e.g. WG, SG, GR, FG, GG, MG), insecticidal articles (e.g. LN), as well as gel formulations for the treatment of plant propagation materials such as seeds (e.g. GF). These and further compositions types are defined in the “Catalogue of pesticide formulation types and international coding system”, Technical Monograph No. 2, 6th Ed. May 2008, CropLife International.

The compositions are prepared in a known manner, such as described by Mollet and Grubemann, Formulation technology, Wiley VCH, Weinheim, 2001; or Knowles, New developments in crop protection product formulation, Agrow Reports DS243, T&F Informa, London, 2005.

Examples for suitable auxiliaries are solvents, liquid carriers, solid carriers or fillers, surfactants, dispersants, emulsifiers, wetters, adjuvants, solubilizers, penetration enhancers, protective colloids, adhesion agents, thickeners, humectants, repellents, attractants, feeding stimulants, compatibilizers, bactericides, anti-freezing agents, anti-foaming agents, colorants, tackifiers and binders.

Suitable solvents and liquid carriers are water and organic solvents, such as mineral oil fractions of medium to high boiling point, e.g. kerosene, diesel oil; oils of vegetable or animal origin; aliphatic, cyclic and aromatic hydrocarbons, e. g. toluene, paraffin, tetrahydronaphthalene, alkylated naphthalenes; alcohols, e.g. ethanol, propanol, butanol, benzylalcohol, cyclohexanol; glycols; DMSO; ketones, e.g. cyclohexanone; esters, e.g. lactates, carbonates, fatty acid esters, gamma-butyrolactone; fatty acids; phosphonates; amines; amides, e.g. N-methylpyrrolidone, fatty acid dimethylamides; and mixtures thereof.

Suitable solid carriers or fillers are mineral earths, e.g. silicates, silica gels, talc, kaolins, limestone, lime, chalk, clays, dolomite, diatomaceous earth, bentonite, calcium sulfate, magnesium sulfate, magnesium oxide; polysaccharide powders, e.g. cellulose, starch; fertilizers, e.g. ammonium sulfate, ammonium phosphate, ammonium nitrate, ureas; products of vegetable origin, e.g. cereal meal, tree bark meal, wood meal, nutshell meal, and mixtures thereof.

Suitable surfactants are surface-active compounds, such as anionic, cationic, nonionic and amphoteric surfactants, block polymers, polyelectrolytes, and mixtures thereof. Such surfactants can be used as emusifier, dispersant, solubilizer, wetter, penetration enhancer, protective colloid, or adjuvant. Examples of surfactants are listed in McCutcheon's, Vol. 1: Emulsifiers & Detergents, McCutcheon's Directories, Glen Rock, USA, 2008 (International Ed. or North American Ed.).

Suitable anionic surfactants are alkali, alkaline earth or ammonium salts of sulfonates, sulfates, phosphates, carboxylates, and mixtures thereof. Examples of sulfonates are alkylaryl-sulfonates, diphenylsulfonates, alpha-olefin sulfonates, lignine sulfonates, sulfonates of fatty acids and oils, sulfonates of ethoxylated alkylphenols, sulfonates of alkoxylated arylphenols, sulfonates of condensed naphthalenes, sulfonates of dodecyl- and tridecylbenzenes, sulfonates of naphthalenes and alkylhnaphthalenes, sulfosuccinates or sulfosuccinamates. Examples of sulfates are sulfates of fatty acids and oils, of ethoxylated alkylphenols, of alcohols, of ethoxylated alcohols, or of fatty acid esters. Examples of phosphates are phosphate esters. Exam-ples of carboxylates are alkyl carboxylates, and carboxylated alcohol or alkylphenol eth-oxylates.

Suitable nonionic surfactants are alkoxylates, N-subsituted fatty acid amides, amine oxides, esters, sugar-based surfactants, polymeric surfactants, and mixtures thereof. Examples of alkoxylates are compounds such as alcohols, alkylphenols, amines, amides, arylphenols, fatty acids or fatty acid esters which have been alkoxylated with 1 to 50 equivalents. Ethylene oxide and/or propylene oxide may be employed for the alkoxylation, preferably ethylene oxide. Examples of N-subsititued fatty acid amides are fatty acid glucamides or fatty acid alkanolamides. Examples of esters are fatty acid esters, glycerol esters or monoglycerides. Examples of sugar-based surfactants are sorbitans, ethoxylated sorbitans, sucrose and glucose esters or alkylpolyglucosides. Examples of polymeric surfactants are homo- or copolymers of vinylpyrrolidone, vinylalcohols, or vinylacetate.

Suitable cationic surfactants are quaternary surfactants, for example quaternary ammonium compounds with one or two hydrophobic groups, or salts of long-chain primary amines. Suitable amphoteric surfactants are alkylbetains and imidazolines. Suitable block polymers are block polymers of the A-B or A-B-A type comprising blocks of polyethylene oxide and polypropylene oxide, or of the A-B—C type comprising alkanol, polyethylene oxide and polypropylene oxide. Suitable polyelectrolytes are polyacids or polybases. Examples of polyacids are alkali salts of polyacrylic acid or polyacid comb polymers. Examples of polybases are polyvinylamines or polyethyleneamines.

Suitable adjuvants are compounds, which have a neglectable or even no pesticidal activity themselves, and which improve the biological performance of the compounds of the present invention on the target. Examples are surfactants, mineral or vegetable oils, and other auxilaries.

Further examples are listed by Knowles, Adjuvants and additives, Agrow Reports DS256, T&F Informa UK, 2006, chapter 5.

Suitable thickeners are polysaccharides (e.g. xanthan gum, carboxymethylcellulose), anorganic clays (organically modified or unmodified), polycarboxylates, and silicates.

Suitable bactericides are bronopol and isothiazolinone derivatives such as alkylisothiazolinones and benzisothiazolinones.

Suitable anti-freezing agents are ethylene glycol, propylene glycol, urea and glycerin.

Suitable anti-foaming agents are silicones, long chain alcohols, and salts of fatty acids.

Suitable colorants (e.g. in red, blue, or green) are pigments of low water solubility and water-soluble dyes. Examples are inorganic colorants (e.g. iron oxide, titan oxide, iron hexacyanoferrate) and organic colorants (e.g. alizarin-, azo- and phthalocyanine colorants).

Suitable tackifiers or binders are polyvinylpyrrolidons, polyvinylacetates, polyvinyl alcohols, polyacrylates, biological or synthetic waxes, and cellulose ethers.

Examples for composition types and their preparation are:

i) Water-Soluble Concentrates (SL, LS)

10-60 wt % of a compound I according to the invention and 5-15 wt % wetting agent (e.g. alcohol alkoxylates) are dissolved in water and/or in a water-soluble solvent (e.g. alcohols) up to 100 wt %. The active substance dissolves upon dilution with water.

ii) Dispersible Concentrates (DC)

5-25 wt % of a compound I according to the invention and 1-10 wt % dispersant (e. g. polyvinylpyrrolidone) are dissolved in up to 100 wt % organic solvent (e.g. cyclohexanone). Dilution with water gives a dispersion.

iii) Emulsifiable Concentrates (EC)

15-70 wt % of a compound I according to the invention and 5-10 wt % emulsifiers (e.g. calcium dodecylbenzenesulfonate and castor oil ethoxylate) are dissolved in up to 100 wt % water-insoluble organic solvent (e.g. aromatic hydrocarbon). Dilution with water gives an emulsion.

iv) Emulsions (EW, EO, ES)

5-40 wt % of a compound I according to the invention and 1-10 wt % emulsifiers (e.g. calcium dodecylbenzenesulfonate and castor oil ethoxylate) are dissolved in 20-40 wt % water-insoluble organic solvent (e.g. aromatic hydrocarbon). This mixture is introduced into up to 100 wt % water by means of an emulsifying machine and made into a homogeneous emulsion. Dilution with water gives an emulsion.

v) Suspensions (SC, OD, FS)

In an agitated ball mill, 20-60 wt % of a compound I according to the invention are comminuted with addition of 2-10 wt % dispersants and wetting agents (e.g. sodium lignosulfonate and alcohol ethoxylate), 0.1-2 wt % thickener (e.g. xanthan gum) and up to 100 wt % water to give a fine active substance suspension. Dilution with water gives a stable suspension of the active substance. For FS type composition up to 40 wt % binder (e.g. polyvinylalcohol) is added.

vi) Water-Dispersible Granules and Water-Soluble Granules (WG, SG)

50-80 wt % of a compound I according to the invention are ground finely with addition of up to 100 wt % dispersants and wetting agents (e.g. sodium lignosulfonate and alcohol ethoxylate) and prepared as water-dispersible or water-soluble granules by means of technical appliances (e. g. extrusion, spray tower, fluidized bed). Dilution with water gives a stable dispersion or solution of the active substance.

vii) Water-Dispersible Powders and Water-Soluble Powders (WP, SP, WS)

50-80 wt % of a compound I according to the invention are ground in a rotor-stator mill with addition of 1-5 wt % dispersants (e.g. sodium lignosulfonate), 1-3 wt % wetting agents (e.g. alcohol ethoxylate) and up to 100 wt % solid carrier, e.g. silica gel. Dilution with water gives a stable dispersion or solution of the active substance.

viii) Gel (GW, GF)

In an agitated ball mill, 5-25 wt % of a compound I according to the invention are comminuted with addition of 3-10 wt % dispersants (e.g. sodium lignosulfonate), 1-5 wt % thickener (e.g. carboxymethylcellulose) and up to 100 wt % water to give a fine suspension of the active substance. Dilution with water gives a stable suspension of the active substance.

ix) Microemulsion (ME)

5-20 wt % of a compound I according to the invention are added to 5-30 wt % organic solvent blend (e.g. fatty acid dimethylamide and cyclohexanone), 10-25 wt % surfactant blend (e.g. alkohol ethoxylate and arylphenol ethoxylate), and water up to 100%. This mixture is stirred for 1 h to produce spontaneously a thermodynamically stable microemulsion.

x) Microcapsules (CS)

An oil phase comprising 5-50 wt % of a compound I according to the invention, 0-40 wt % water insoluble organic solvent (e.g. aromatic hydrocarbon), 2-15 wt % acrylic monomers (e.g. methylmethacrylate, methacrylic acid and a di- or triacrylate) are dispersed into an aqueous solution of a protective colloid (e.g. polyvinyl alcohol). Radical polymerization initiated by a radi-cal initiator results in the formation of poly(meth)acrylate microcapsules. Alternatively, an oil phase comprising 5-50 wt % of a compound I according to the invention, 0-40 wt % water insolu-ble organic solvent (e.g. aromatic hydrocarbon), and an isocyanate monomer (e.g. diphenylme-thene-4,4′-diisocyanatae) are dispersed into an aqueous solution of a protective colloid (e.g. polyvinyl alcohol). The addition of a polyamine (e.g. hexamethylenediamine) results in the for-mation of a polyurea microcapsule. The monomers amount to 1-10 wt %. The wt % relate to the total CS composition.

xi) Dustable Powders (DP, DS)

1-10 wt % of a compound I according to the invention are ground finely and mixed intimately with up to 100 wt % solid carrier, e.g. finely divided kaolin.

xii) Granules (GR, FG)

0.5-30 wt % of a compound I according to the invention is ground finely and associated with up to 100 wt % solid carrier (e.g. silicate). Granulation is achieved by extrusion, spray-drying or the fluidized bed.

xiii) Ultra-Low Volume Liquids (UL)

1-50 wt % of a compound I according to the invention are dissolved in up to 100 wt % organic solvent, e.g. aromatic hydrocarbon.

The compositions types i) to xi) may optionally comprise further auxiliaries, such as 0.1-1 wt % bactericides, 5-15 wt % anti-freezing agents, 0.1-1 wt % anti-foaming agents, and 0.1-1 wt % colorants.

The agrochemical compositions generally comprise between 0.01 and 95%, preferably between 0.1 and 90%, and most preferably between 0.5 and 75%, by weight of active sub-stance. The active substances are employed in a purity of from 90% to 100%, preferably from 95% to 100% (according to NMR spectrum).

Various types of oils, wetters, adjuvants, fertilizer, or micronutrients, and other pesticides (e.g. herbicides, insecticides, fungicides, growth regulators, safeners) may be added to the active substances or the compositions com-prising them as premix or, if appropriate not until immediately prior to use (tank mix). These agents can be admixed with the compositions according to the invention in a weight ratio of 1:100 to 100:1, preferably 1:10 to 10:1.

The user applies the composition according to the invention usually from a predosage de-vice, a knapsack sprayer, a spray tank, a spray plane, or an irrigation system. Usually, the agrochemical composition is made up with water, buffer, and/or further auxiliaries to the desired application concentration and the ready-to-use spray liquor or the agrochemical composition according to the invention is thus obtained. Usually, 20 to 2000 liters, preferably 50 to 400 liters, of the ready-to-use spray liquor are applied per hectare of agricultural useful area.

According to one embodiment, individual components of the composition according to the invention such as parts of a kit or parts of a binary or ternary mixture may be mixed by the user himself in a spray tank and further auxiliaries may be added, if appropriate.

In a further embodiment, either individual components of the composition according to the invention or partially premixed components, e. g. components comprising compounds of the present invention and/or mixing partners as defined above, may be mixed by the user in a spray tank and further auxiliaries and additives may be added, if appropriate.

In a further embodiment, either individual components of the composition according to the invention or partially premixed components, e. g. components comprising compounds of the present invention and/or mixing partners as defined above, can be applied jointly (e.g. after tank mix) or consecutively.

The compounds of the present invention are suitable for use in protecting crops, plants, plant propagation materials, such as seeds, or soil or water, in which the plants are growing, from attack or infestation by animal pests. Therefore, the present invention also relates to a plant protection method, which comprises contacting crops, plants, plant propagation materials, such as seeds, or soil or water, in which the plants are growing, to be protected from attack or infestation by animal pests, with a pesticidally effective amount of a compound of the present invention.

The compounds of the present invention are also suitable for use in combating or controlling animal pests. Therefore, the present invention also relates to a method of combating or controlling animal pests, which comprises contacting the animal pests, their habitat, breeding ground, or food supply, or the crops, plants, plant propagation materials, such as seeds, or soil, or the area, material or environment in which the animal pests are growing or may grow, with a pesticidally effective amount of a compound of the present invention.

The compounds of the present invention are effective through both contact and ingestion. Furthermore, the compounds of the present invention can be applied to any and all developmental stages, such as egg, larva, pupa, and adult.

The compounds of the present invention can be applied as such or in form of compositions comprising them as defined above. Furthermore, the compounds of the present invention can be applied together with a mixing partner as defined above or in form of compositions comprising said mixtures as defined above. The components of said mixture can be applied simultaneously, jointly or separately, or in succession, that is immediately one after another and thereby creating the mixture “in situ” on the desired location, e.g. the plant, the sequence, in the case of separate application, generally not having any effect on the result of the control measures.

The application can be carried out both before and after the infestation of the crops, plants, plant propagation materials, such as seeds, soil, or the area, material or environment by the pests.

Suitable application methods include inter alia soil treatment, seed treatment, in furrow application, and foliar application. Soil treatment methods include drenching the soil, drip irrigation (drip application onto the soil), dipping roots, tubers or bulbs, or soil injection. Seed treatment techniques include seed dressing, seed coating, seed dusting, seed soaking, and seed pelleting. In furrow applications typically include the steps of making a furrow in cultivated land, seeding the furrow with seeds, applying the pesticidally active compound to the furrow, and closing the furrow. Foliar application refers to the application of the pesticidally active compound to plant foliage, e.g. through spray equipment. For foliar applications, it can be advantageous to modify the behavior of the pests by use of pheromones in combination with the compounds of the present invention. Suitable pheromones for specific crops and pests are known to a skilled person and publicly available from databases of pheromones and semiochemicals, such as http://www.pherobase.com.

As used herein, the term “contacting” includes both direct contact (applying the compounds or compositions directly on the animal pest or plant—typically to the foliage, stem or roots of the plant) and indirect contact (applying the compounds/compositions to the locus, i.e. habitat, breeding ground, plant, seed, soil, area, material or environment in which a pest is growing or may grow, of the animal pest or plant).

The term “animal pest” includes arthropods, gastropods, and nematodes. Preferred animal pests according to the invention are arthropods, preferably insects and arachnids, in particular insects. Insects, which are of particular relevance for crops, are typically referred to as crop insect pests.

The term “crop” refers to both, growing and harvested crops.

The term “plant” includes cereals, e.g. durum and other wheat, rye, barley, triticale, oats, rice, or maize (fodder maize and sugar maize/sweet and field corn); beet, e.g. sugar beet or fodder beet; fruits, such as pomes, stone fruits or soft fruits, e.g. apples, pears, plums, peaches, nectarines, almonds, cherries, papayas, strawberries, raspberries, blackberries or gooseberries; leguminous plants, such as beans, lentils, peas, alfalfa or soybeans; oil plants, such as rapeseed (oilseed rape), turnip rape, mustard, olives, sunflowers, coconut, cocoa beans, castor oil plants, oil palms, ground nuts or soybeans; cucurbits, such as squashes, pumpkins, cucumber or melons; fiber plants, such as cotton, flax, hemp or jute; citrus fruit, such as oranges, lemons, grapefruits or mandarins; vegetables, such as eggplant, spinach, lettuce (e.g. iceberg lettuce), chicory, cabbage, asparagus, cabbages, carrots, onions, garlic, leeks, tomatoes, potatoes, cucurbits or sweet peppers; lauraceous plants, such as avocados, cinnamon or camphor; energy and raw material plants, such as corn, soybean, rapeseed, sugar cane or oil palm; tobacco; nuts, e.g. walnuts; pistachios; coffee; tea; bananas; vines (table grapes and grape juice grape vines); hop; sweet leaf (also called Stevia); natural rubber plants or ornamental and forestry plants, such as flowers (e.g. carnation, petunias, geranium/pelargoniums, pansies and impatiens), shrubs, broad-leaved trees (e.g. poplar) or evergreens, e.g. conifers; eucalyptus; turf; lawn; grass such as grass for animal feed or ornamental uses. Preferred plants include potatoes sugar beets, tobacco, wheat, rye, barley, oats, rice, corn, cotton, soybeans, rapeseed, legumes, sunflowers, coffee or sugar cane; fruits; vines; ornamentals; or vegetables, such as cucumbers, tomatoes, beans or squashes.

The term “plant” is to be understood as including plants, which have been modified by either conventional breeding, or mutagenesis or genetic engineering, or by a combination thereof.

Plants, which have been modified by mutagenesis or genetic engineering, and are of particular commercial importance, include alfalfa, rapeseed (e.g. oilseed rape), bean, carnation, chicory, cotton, eggplant, eucalyptus, flax, lentil, maize, melon, papaya, petunia, plum, poplar, potato, rice, soybean, squash, sugar beet, sugarcane, sunflower, sweet pepper, tobacco, tomato, and cereals (e.g. wheat), in particular maize, soybean, cotton, wheat, and rice. In plants, which have been modified by mutagenesis or genetic engineering, one or more genes have been mutagenized or integrated into the genetic material of the plant. The one or more mutagenized or integrated genes are preferably selected from pat, epsps, cry1Ab, bar, cry1Fa2, cry1Ac, cry34Ab1, cry35AB1, cry3A, cryF, cry1F, mcry3a, cry2Ab2, cry3Bbl, cry1A.105, dfr, barnase, vip3Aa20, barstar, als, bxn, bp40, asn1, and ppo5. The mutagenesis or integration of the one or more genes is performed in order to improve certain properties of the plant. Such properties, also known as traits, include abiotic stress tolerance, altered growth/yield, disease resistance, herbicide tolerance, insect resistance, modified product quality, and pollination control. Of these properties, herbicide tolerance, e.g. imidazolinone tolerance, glyphosate tolerance, or glufosinate tolerance, is of particular importance. Several plants have been rendered tolerant to herbicides by mutagenesis, for example Clearfield® oilseed rape being tolerant to imidazolinones, e.g. imazamox. Alternatively, genetic engineering methods have been used to render plants, such as soybean, cotton, corn, beets and oil seed rape, tolerant to herbicides, such as glyphosate and glufosinate, some of which are commercially available under the trade names RoundupReady® (glyphosate) and LibertyLink® (glufosinate). Furthermore, insect resistance is of importance, in particular lepidopteran insect resistance and coleopteran insect resistance. Insect resistance is typically achieved by modifying plants by integrating cry and/or vip genes, which were isolated from Bacillus thuringiensis (Bt), and code for the respective Bt toxins. Genetically modified plants with insect resistance are commercially available under trade names including WideStrike®, Bollgard®, Agrisure®, Herculex®, YieldGard®, Genuity®, and Intacta®. Plants may be modified by mutagenesis or genetic engineering either in terms of one property (singular traits) or in terms of a combination of properties (stacked traits). Stacked traits, e.g. the combination of herbicide tolerance and insect resistance, are of increasing importance. In general, all relevant modified plants in connection with singular or stacked traits as well as detailed information as to the mutagenized or integrated genes and the respective events are available from websites of the organizations “International Service for the Acquisition of Agri-biotech Applications (ISAAA)” (http://www.isaaa.org/gmapprovaldatabase) and “Center for Environmental Risk Assessment (CERA)” (http://cera-gmc.org/GMCropDatabase).

It has surprisingly been found that the pesticidal activity of the compounds of the invention may be enhanced by the insecticidal trait of a modified plant. Furthermore, it has been found that the compounds of the present invention are suitable for preventing insects to become resistant to the insecticidal trait or for combating pests, which already have become resistant to the insecticidal trait of a modified plant. Moreover, the compounds of the present invention are suitable for combating pests, against which the insecticidal trait is not effective, so that a complementary insecticidal activity can advantageously be used.

The term “plant propagation material” refers to all the generative parts of the plant such as seeds and vegetative plant material such as cuttings and tubers (e.g. potatoes), which can be used for the multiplication of the plant. This includes seeds, roots, fruits, tubers, bulbs, rhizomes, shoots, sprouts and other parts of plants. Seedlings and young plants, which are to be transplanted after germination or after emergence from soil, may also be included. These plant propagation materials may be treated prophylactically with a plant protection compound either at or before planting or transplanting.

The term “seed” embraces seeds and plant propagules of all kinds including but not limited to true seeds, seed pieces, suckers, corms, bulbs, fruit, tubers, grains, cuttings, cut shoots and the like, and means in a preferred embodiment true seeds.

In general, “pesticidally effective amount” means the amount of active ingredient needed to achieve an observable effect on growth, including the effects of necrosis, death, retardation, prevention, and removal, destruction, or otherwise diminishing the occurrence and activity of the target organism. The pesticidally effective amount can vary for the various compounds/compositions used in the invention. A pesticidally effective amount of the compositions will also vary according to the prevailing conditions such as desired pesticidal effect and duration, weather, target species, locus, mode of application, and the like.

In the case of soil treatment, in furrow application or of application to the pests dwelling place or nest, the quantity of active ingredient ranges from 0.0001 to 500 g per 100 m², preferably from 0.001 to 20 g per 100 m².

For use in treating crop plants, e.g. by foliar application, the rate of application of the active ingredients of this invention may be in the range of 0.0001 g to 4000 g per hectare, e.g. from 1 g to 2 kg per hectare or from 1 g to 750 g per hectare, desirably from 1 g to 100 g per hectare, more desirably from 10 g to 50 g per hectare, e.g., 10 to 20 g per hectare, 20 to 30 g per hectare, 30 to 40 g per hectare, or 40 to 50 g per hectare.

The compounds of the present invention are particularly suitable for use in the treatment of seeds in order to protect the seeds from insect pests, in particular from soil-living insect pests, and the resulting seedling's roots and shoots against soil pests and foliar insects. The present invention therefore also relates to a method for the protection of seeds from insects, in particular from soil insects, and of the seedling's roots and shoots from insects, in particular from soil and foliar insects, said method comprising treating the seeds before sowing and/or after pregermination with a compound of the present invention. The protection of the seedling's roots and shoots is preferred. More preferred is the protection of seedling's shoots from piercing and sucking insects, chewing insects and nematodes.

The term “seed treatment” comprises all suitable seed treatment techniques known in the art, such as seed dressing, seed coating, seed dusting, seed soaking, seed pelleting, and in-furrow application methods. Preferably, the seed treatment application of the active compound is carried out by spraying or by dusting the seeds before sowing of the plants and before emergence of the plants.

The present invention also comprises seeds coated with or containing the active compound. The term “coated with and/or containing” generally signifies that the active ingredient is for the most part on the surface of the propagation product at the time of application, although a greater or lesser part of the ingredient may penetrate into the propagation product, depending on the method of application. When the said propagation product is (re)planted, it may absorb the active ingredient.

Suitable seed is for example seed of cereals, root crops, oil crops, vegetables, spices, ornamentals, for example seed of durum and other wheat, barley, oats, rye, maize (fodder maize and sugar maize/sweet and field corn), soybeans, oil crops, crucifers, cotton, sunflowers, bananas, rice, oilseed rape, turnip rape, sugarbeet, fodder beet, eggplants, potatoes, grass, lawn, turf, fodder grass, tomatoes, leeks, pumpkin/squash, cabbage, iceberg lettuce, pepper, cucumbers, melons, Brassica species, melons, beans, peas, garlic, onions, carrots, tuberous plants such as potatoes, sugar cane, tobacco, grapes, petunias, geranium/pelargoniums, pansies and impatiens.

In addition, the active compound may also be used for the treatment of seeds from plants, which have been modified by mutagenisis or genetic engineering, and which e.g. tolerate the action of herbicides or fungicides or insecticides. Such modified plants have been described in detail above.

Conventional seed treatment formulations include for example flowable concentrates FS, solutions LS, suspoemulsions (SE), powders for dry treatment DS, water dispersible powders for slurry treatment WS, water-soluble powders SS and emulsion ES and EC and gel formulation GF. These formulations can be applied to the seed diluted or undiluted. Application to the seeds is carried out before sowing, either directly on the seeds or after having pregerminated the latter. Preferably, the formulations are applied such that germination is not included.

The active substance concentrations in ready-to-use formulations, which may be obtained after two-to-tenfold dilution, are preferably from 0.01 to 60% by weight, more preferably from 0.1 to 40% by weight.

In a preferred embodiment a FS formulation is used for seed treatment. Typically, a FS formulation may comprise 1-800 g/l of active ingredient, 1-200 g/l Surfactant, 0 to 200 g/l antifreezing agent, 0 to 400 g/l of binder, 0 to 200 g/l of a pigment and up to 1 liter of a solvent, preferably water.

Especially preferred FS formulations of the compounds of the present invention for seed treatment usually comprise from 0.1 to 80% by weight (1 to 800 g/l) of the active ingredient, from 0.1 to 20% by weight (1 to 200 g/1) of at least one surfactant, e.g. 0.05 to 5% by weight of a wetter and from 0.5 to 15% by weight of a dispersing agent, up to 20% by weight, e.g. from 5 to 20% of an anti-freeze agent, from 0 to 15% by weight, e.g. 1 to 15% by weight of a pigment and/or a dye, from 0 to 40% by weight, e.g. 1 to 40% by weight of a binder (sticker/adhesion agent), optionally up to 5% by weight, e.g. from 0.1 to 5% by weight of a thickener, optionally from 0.1 to 2% of an anti-foam agent, and optionally a preservative such as a biocide, antioxidant or the like, e.g. in an amount from 0.01 to 1% by weight and a filler/vehicle up to 100% by weight.

In the treatment of seed, the application rates of the compounds of the invention are generally from 0.1 g to 10 kg per 100 kg of seed, preferably from 1 g to 5 kg per 100 kg of seed, more preferably from 1 g to 1000 g per 100 kg of seed and in particular from 1 g to 200 g per 100 kg of seed, e.g. from 1 g to 100 g or from 5 g to 100 g per 100 kg of seed.

The invention therefore also relates to seed comprising a compound of the present invention, or an agriculturally useful salt thereof, as defined herein. The amount of the compound of the present invention or the agriculturally useful salt thereof will in general vary from 0.1 g to 10 kg per 100 kg of seed, preferably from 1 g to 5 kg per 100 kg of seed, in particular from 1 g to 1000 g per 100 kg of seed. For specific crops such as lettuce the rate can be higher.

### EXAMPLES

The present invention is now illustrated in further details by the following example, without imposing any limitation thereto.

The biological activity and effectivity of the compounds applied in the methods of the invention can be evaluated e.g. in the following assay.

Action on Brown Stink Bug (Euschistus heros)

The active compound is dissolved at the desired concentration in a mixture of 1:1 (vol:vol) distilled water: acetone.

Soybean seedlings of Intacta® and BMX-Potencia with one pair of true leaves were sprayed with 2 ml solution using an airbrush. After dry, the plants in small pots were placed inside a transparent plastic cage and infested with 10 adults adult stage individuals. Treated cups were kept at about 25° C. Percent mortality was recorded after 3 and 7 days after application.

In this test, Compound I.1 at 10 and 1 ppm showed the following mortality:

| | | | | |
|---|---|---|---|---|
| | dose | 3 | 7 | mortality (Abbott %)* 7 |
| Product | ppm a.i | DAA | DAA | DAA |

INTACTA ®

| | | | | |
|---|---|---|---|---|
| Untreated | — | 15.0 | 15.0 | — |
| I.1 | 10 | 17.5 | 85.0 | 82.4 |
| I.1 | 1 | 12.5 | 57.5 | 50.0 |

BMX - POTENCIA

| | | | | |
|---|---|---|---|---|
| Untreated | — | 12.5 | 12.5 | — |
| I.1 | 10 | 17.5 | 82.5 | 80.0 |
| I.1 | 1 | 7.5 | 50.0 | 42.9 |

DAA = Days After Application
*Corrected mortality according to Abbott, W. S., A method of computing the effectiveness of an insecticide, J. Econ. Entomal., 18 (1925) 265-267.

The test shows that the compound of formula I has a higher efficacy when applied to soybean plants containing a toxin trait of Bacillus thuringiensis (INTACTA®) than when applied to genetically modified soybean plants without the Bacillus thuringiensis trait (BMX-POTENCIA). This is surprising since the values in the untreated groups (corrected according to Abbott) are equal, i.e. the Bacillus thuringiensis toxin alone does not control brown stink bugs.

1-15. (canceled)

16. A method for controlling pests of soybean, corn and cotton plants, wherein the plants are Bt plants, comprising the step of contacting the plant, parts of it, its propagation material, the pests, their food supply, habitat, or breeding grounds with one or more compounds of formula I 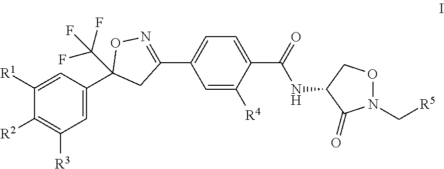
wherein
R¹ is Cl, or CF₃;
R² is F, or H;
R³ is Cl, or H;
R⁴ is CH₃, or Cl; and
R⁵ is H, CH₃, or CF₃,
or a salt or N-oxide thereof.

17. The method according to claim 16, wherein the compounds of formula I are selected from compounds of formula I.1 and I.2

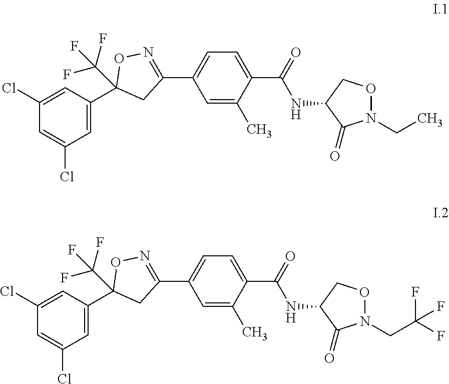 [SMILES: CCN1OC[C@@H](NC(=O)c2ccc(C3=NOC(c4cc(Cl)cc(Cl)c4)(C(F)(F)F)C3)cc2C)C1=O ; Cc1cc(C2=NOC(c3cc(Cl)cc(Cl)c3)(C(F)(F)F)C2)ccc1C(=O)N[C@@H]1CON(CC(F)(F)F)C1=O]

18. A method for controlling pests of soybean, corn, and cotton plants, comprising the step of contacting the plant, parts of it, its propagation material, the pests, their food supply, habitat or breeding grounds with composition comprising a compound of formula I as defined in claim 16 and one or more other pesticides as mixing partners.

19. The method according to claim 16 wherein the plants are geneticaly modified plants selected from soybean, corn, and cotton.

20. The method according to claim 16 wherein the plants are soybean plants.

21. The method according to claim 16, wherein the pests are Acrosternum spp., Euschistus spp., Nezara spp., Piezodrus spp., Euschistus heros, Euschistus impictiventris, Euschistus variolarius, Euschistus servus, Nezara viridula, Piezodorus guildinii, Halyomorpha halys, Euschistus servus, Euschistus variolarius, Halyomorpha halys, Lygus hesperus, Lygus lineolaris, and/or Lygus pratensis.

22. The method according to claim 16, wherein the pests are Acrosternum hilare, Euschistus heros, Nezara viridula, and/or Piezodorus guildini.

23. The method according to claim 16, wherein the pests are Euschistus heros.

24. The method according to claim 16, wherein the compounds of formula I or a composition comprising the compound of formula I are applied in an amount of from 1 to 500 g/ha.

25. The method according to claim 16 for protecting plant propagation material.

26. A method for controlling pests from the family of Pentatomidae and/or Thripidae or Miridae, comprising the step of contacting the pests, their food supply, habitat and/or breeding ground with one or more compounds of formula I as defined in claim 16, or a composition comprising compound of formula I as defined in claim 16.

27. The method according to claim 16, wherein the pests are Pentatomidae and are Acrosternum spp. such as Acrosternum hilare, Euschistus spp. such as Euschistus heros, Euschistus impictiventris, Euschistus variolarius, Euschistus servus, Nezara spp. such as Nezara viridula and/or Piezodrus spp. such as Piezodrus guildini, Halyomorpha halys, and/or Miridae, such as Lygus hesperus, Lygus lineolaris and Lygus pratensis.

28. The method according to claim 16, wherein the pests are Euschistus spp. such as Euschistus heros, Euschistus impictiventris, Euschistus variolarius, and Euschistus servus.

29. The method according to claim 16, wherein the compound of formula I or a composition comprising compound of formula I are applied in an amount of from 1 to 500 g/ha.

30. The use of one or more compounds of formula I as defined in claim 16 for controlling pests from the family of Pentatomidae and/or Thripidae and/or Miridae in genetically modified soybean, corn, and cotton crops according to claim 16.
